(19) United States
(12) Patent Application Publication
(10) Pub. No.: US 2016/0082263 A1
(43) Pub. Date: Mar. 24, 2016

(54) SYSTEM AND METHOD FOR DETERMINING OPTIMAL DEEP BRAIN STIMULATION PARAMETERS FOR TREATING INTRACTABLE EPILEPSY

(71) Applicant: Harinder JASEJA, Gwalior (IN)

(72) Inventor: Harinder JASEJA, Gwalior (IN)

(21) Appl. No.: 14/556,259

(22) Filed: Dec. 1, 2014

(30) Foreign Application Priority Data
Sep. 18, 2014 (IN) 2986/MUM/2014

(51) Int. Cl. A61N 1/36

(52) U.S. Cl. CPC A61N 1/36139; A61N 1/36064; A61N 1/36175; A61N 1/0534

(57) ABSTRACT

System and method for determining optimal deep brain stimulation parameters for treating intractable epilepsy. The system is adapted to impart deep brain stimulation to a patient's brain, record electroencephalographic responses to the stimulation, enable adjustment of one or more stimulation parameters to achieve an optimum electroencephalogram desynchronization (EEG desynchronization) state and thereafter select stimulation parameters that resulted in the optimum EEG desynchronization.

The following specification particularly describes the invention and the manner in which it is to be performed.

### FIELD

In general, the subject matter relates to the field of treatment of intractable epilepsy. More particularly, but not exclusively, to deep brain stimulation of the brain to treat intractable epilepsy.

### DISCUSSION OF RELATED FIELD

Deep brain stimulation (hereinafter known as DES) involves stimulating the patient's brain by sending electrical impulses through electrical leads implanted in the patient's brain. The electrical impulses are generated by a stimulator, which is configured to be programmed by a programmer to adjust the stimulation parameters.

The success of the DES largely depends on selection of effective stimulation parameters. It is proposed that, effective stimulation parameters are to be selected with the objective of inducing electroencephalogram de-synchronization (hereinafter known as EEG desynchronization). The objective of inducing EEG desynchronization is justified by availability of various studies documenting that EEG desynchronization is strongly associated with marked resistance to seizure activity and attacks with even a suppressive effect on interictal epileptiform discharges (IEDs). EEG desynchronization has been widely proposed as an important mechanism of the antiepileptic action of electrical stimulation techniques like vagal nerve and trigeminal nerve stimulations in intractable epilepsy.

In light of the advantages of imparting DES at optimal stimulation parameters to induce EEG desynchronization. Currently, certain methods are adopted by professionals to determine the optimal stimulation parameters. Such methods involve imparting DES at certain stimulation parameters to the patient's brain and thereafter studying the effects of the DBS over a period of time, to determine if the selected stimulation parameters were able to produce desired relief in the intractable seizures. The selection of stimulation parameters is carried out over multiple sessions based on periodic clinical responses. Such methods involve trial and error processes and also result in multiple hospital visits, before finalization of effective stimulation parameters. Further, the selected stimulation parameters may not even be optimal stimulation parameters. At present, there exist no techniques, which in a single clinical visit, may be able to accurately determine optimal stimulation parameters to induce optimum EEG desynchronization.

In light of the foregoing discussion, there is a need for a technique to accurately determine optimal stimulation parameters that induce optimum EEG desynchronization.

### OBJECTIVE OF THE INVENTION

An objective of the present invention is to determine optimal stimulation parameters for treating intractable epilepsy by deep brain stimulation by achieving an optimum electroencephalogram desynchronization state with a simultaneous EEG recording.

### SUMMARY

In an embodiment, a system is provided for determining optimal stimulation parameters for treating intractable epilepsy by deep brain stimulation. The system is adapted to impart deep brain stimulation to a patient's brain, record EEG responses to the stimulation, enable adjustment of one or more stimulation parameters to achieve an optimum EEG desynchronization state and thereafter select stimulation parameters that resulted in the optimum EEG desynchronization.

In another embodiment, a method is provided for determining optimal stimulation parameters for treating intractable epilepsy by deep brain stimulation. The method includes imparting deep brain stimulation to a patient's brain, recording EEG responses to the stimulation, enabling adjustment of one or more stimulation parameters to achieve an optimum EEG desynchronization state and thereafter selecting stimulation parameters that resulted in the optimum EEG desynchronization.

### BRIEF DESCRIPTION OF DRAWINGS

Embodiments are illustrated by way of example and not limitation in the Figures of the accompanying drawings, in Which like references indicate similar elements and in which:

FIG. 1 illustrates a system for determining optimal stimulation parameters for treating intractable epilepsy in accordance with an embodiment; and

FIG. 2 is an exemplary flow chart for determining optimal stimulation parameters for treating intractable epilepsy in accordance with an embodiment.

### DETAILED DESCRIPTION OF PREFERRED EMBODIMENTS

I. Overview

II. Exemplary System

III. Exemplary Mechanism of Action

IV. Exemplary Method

V. Conclusion

### I. Overview

In general, subject matter relates to treatment of intractable epilepsy, more particularly, but not exclusively, to treatment of epilepsy using stimulation provided by deep brain stimulation. In an embodiment, epilepsy is treated by imparting anterior thalamic nucleus deep brain stimulation (hereinafter referred to as ATN DBS) to a patient suffering with intractable epilepsy. For effective treatment of a patient suffering from intractable epilepsy through ATN DBS, placement of electrodes and providing effective stimulation parameters play important roles, In an embodiment, stimulation parameters are adjusted to achieve electroencephalogram desynchronization (hereinafter referred to as EEG desynchronization) while imparting ATN DBS. The EEG response to adjustments to stimulation parameters can be visible on a simultaneously ongoing EEG recording, which enables identification of optimal stimulation parameters at which EEG desynchronization may be achieved.

The following detailed description includes references to the accompanying drawing, which form part of the detailed description. The drawing shows illustration in accordance with example embodiments. These example embodiments are described in enough detail to enable those skilled in the art to practice the present subject matter. However, it will be apparent to one of ordinary skill in the art that the present invention may be practiced without these specific details. In other instances, well-known methods, procedures and components have not been described in detail so as not to unnecessarily obscure aspects of the embodiments. The embodiments can be combined, other embodiments can be utilized or structural and logical changes can be made without departing from the scope of the invention. The following detailed description is, therefore, not to be taken as a limiting sense.

In this document, the terms “a” or “an” are used, as is common in patent documents, to include one or more than one. In this document, the term “or” is used to refer to a nonexclusive “or,” such that “A or B” includes “A but not B,” “B but not A,” and “A and B,” unless otherwise indicated.

### II. Exemplary System

Epilepsy is considered to occur due to neurological disorders, when the brain activity is in an abnormally synchronized state. Inducing EEG desynchronization can be considered to be an effective way of resisting occurrence of seizure activity in patients suffering from epilepsy. EEG desynchronization may be defined as an EEG pattern lacking regular periodicity in its waveform. EEG desynchronization can be induced by DBS.

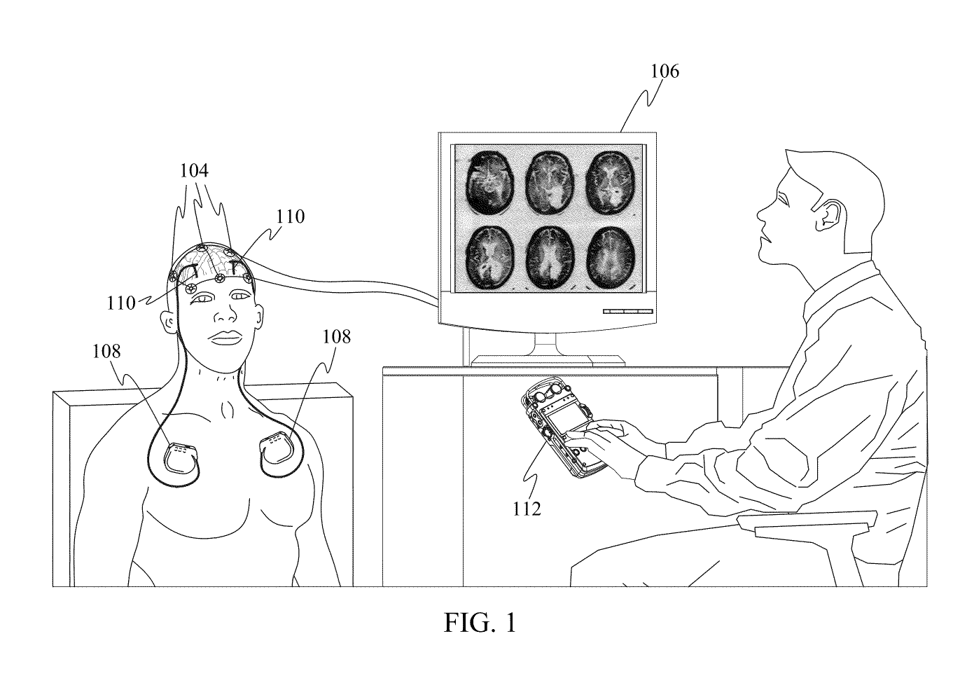

FIG. 1 illustrates a system for determining optimal stimulation parameters for treating intractable epilepsy. The system includes a DBS device 102, EEG headset 104 and an EEG recorder 106. The DBS device 102 includes stimulators 108, leads 110 and a programmer 112. The DBS device 102 is configured to deliver electrical pulses to the patient's brain thorough the leads 110. The stimulators 108 can be configured to generate electrical impulses. The programmer 112 can be configured to adjust the stimulation parameters on the stimulators 108. The stimulation parameters can be the electrical impulses which are generated by the stimulators 108. The stimulators 108 can be embedded under the skin of the patient at a suitable location. Upon adjusting the stimulation parameters, the stimulators 108 deliver electrical impulses to the brain of the patient through the leads 110, Which are embedded in the patient's brain. EEG is the recording of electrical activity along the scalp. Conducting the EEG can detect abnormalities related to electrical activity of the brain. EEG tracks and records wave patterns. The study of the patterns can detect synchronization and desynchronization states of the brain. The EEG headset 104 can be placed over the patient's scalp. The EEG headset 104 includes EEG electrodes, which are stuck to the patients scalp at locations on the scalp which facilitate determination of the patterns. The EEG headset is connected to an EEG machine, which includes an EEG recorder 106. The EEG recorder 106 can be configured to display the EEG patterns. A trained practitioner can study the patterns displayed on the EEG recorder and derive conclusions as to the state of the patient's brain.

The trained practitioner may adjust the stimulation parameters generated by the stimulators 108 using the programmer 112. The stimulators can thereafter provide stimulations to the patient's brain. The EEG can be simultaneously carried out while imparting DBS to the patient's brain. While imparting DBS to the patient's brain, the EEG recorder may record the patterns generated upon delivering different stimulation parameters. Upon studying the patterns the trained practitioner may determine if an optimum EEG desynchronization has been achieved. The stimulation parameters at which the optimum EEG desynchronization is achieved can be considered as optimal stimulation parameters. The optimal stimulation parameters can be programmed to the stimulators 108 using the programmer 112 to achieve optimum EEG desynchronization, thereby reducing the risk of seizures attacks in patients suffering from intractable epilepsy. The risk of side effects to the patient may be minimized by using optimal stimulation parameters to stimulate the patient's brain.

### III. Exemplary Mechanism of Action

In an embodiment, the DBS imparted to the patient can be an anterior thalamic nucleus DBS.

In an embodiment, the DBS imparted to the patient can be a centromedian thalamic nucleus DBS.

Kindling has been known to induce epileptogenesis which may lead to seizure attacks. Desynchronization is also known to reverse the kindling process and thereby aid in treatment of intractable epilepsy. In an embodiment, the optimal stimulation parameters can achieve an anti kindling effect by inducing EEG desynchronization and aid in treatment of intractable epilepsy.

In an embodiment, ATN DBS may be imparted to the patient's brain involving Papez circuit. Techniques of treatment through ATN-DBS would be widened so as to include intractable generalized seizures and Lennox-Gastaut syndrome also in its therapeutic indications.

In an embodiment, the centromedian thalamic nucleus (CMN) DBS may be imparted to the patient's brain involving reticular activating system.

In an embodiment, DBS of various targets in neurological disorders other than intractable epilepsy may also benefit from induction of EEG desynchronization especially in those disorders that are underpinned with abnormal neuronal synchronization like Parkinson's disease.

In an embodiment, the pulse width for inducing EEG desynchronization can be in the range of 110 to 130 microseconds.

In an embodiment, voltages of electrical pulses may be relatively low in accordance with Weiss equation (pulse width×1/voltage).

A total electrical energy delivered to the patient's brain can be expressed by a formula {[(voltage²×pulse width×frequency)/impedance]×1 s}. In an embodiment, the total electrical energy delivered to the patient's brain can be low, thereby minimizing side effects and stimulator battery consumption.

### IV. Exemplary Method

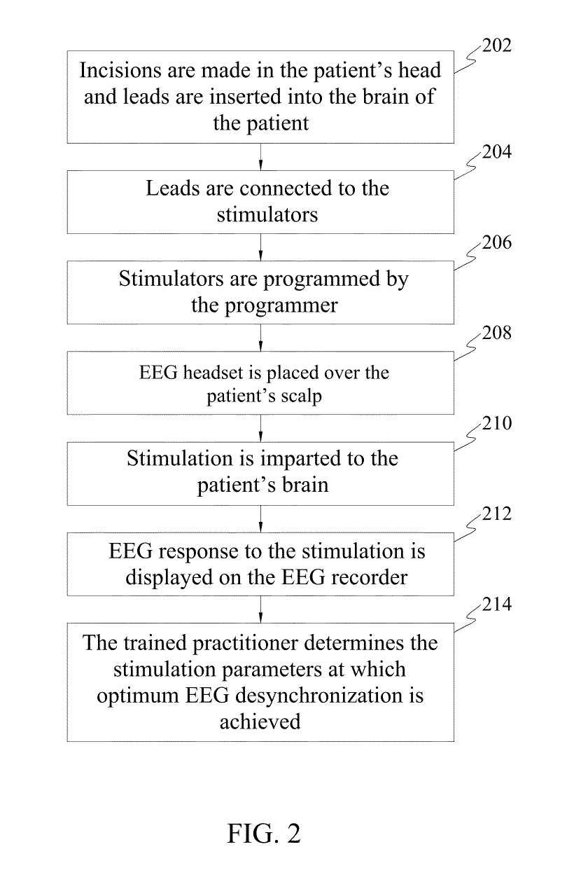

FIG. 2 is an exemplary flow chart for determining optimal stimulation a for treating intractable epilepsy.

At step 202, incisions are made in the patient's head, such that, leads 110 can be inserted into the brain of the patient. At step 204 the leads 110 are connected to the stimulators 108. At step 206, the stimulators 108 are programmed by the programmer 112 to adjust stimulation parameters on the stimulators 108. At step 208, the EEG headset 104 is placed over the patient's scalp. At step 210, stimulation is imparted to the patient's brain. At step 212 the EEG response to the stimulation is displayed on the EEG recorder 106. At step 214, the trained practitioner studies the patterns on the EEG recorder 106 to determine the stimulation parameters at which optimum EEG desynchronization is achieved. The stimulation parameters at which optimum EEG desynchronization is achieved can be considered as optimal stimulation parameters.

### V. CONCLUSION

In light of the above disclosure, it is evident that, the present invention has many advantages over existing technologies. Some of those advantages are mentioned below:

The instant technique of determining optimal stimulation parameters for achieving optimum EEG desynchronization facilitates determination of the optimal stimulation parameters even at the first session, unlike the techniques of prior art, wherein the stimulation parameters are determined over a period of time based on periodic clinical responses to stimulation provided to the patient.

The sessions required are minimized compared to sessions required for techniques of prior art.

Uncertainties concerning the success of DBS may be eliminated.

The values of stimulation parameters can be kept minimal as required to induce EEG desynchronization, thereby minimizing side effects and stimulator battery consumption.

The processes described above are described as sequence of steps; this was done solely for the sake of illustration. Accordingly, it is contemplated that some steps may be added, some steps may be omitted, the order of the steps may be re-arranged, or some steps may be performed simultaneously.

Although embodiments have been described with reference to specific example embodiments, it will be evident that various modifications and changes may be made to these embodiments without departing from the broader spirit and scope of the system and method described herein. Accordingly, the specification and drawings are to be regarded in an illustrative rather than a restrictive sense.

Many alterations and modifications of the present invention will no doubt become apparent to a person of ordinary skill in the art after having read the foregoing description. It is to be understood that the phraseology or terminology employed herein is for the purpose of description and not of limitation. It is to be understood that the description above contains many specifications; these should not be construed as limiting the scope of the invention but as merely providing illustrations of some of the personally preferred embodiments of this invention. Thus the scope of the invention should be determined by the appended claims and their legal equivalents rather than by the examples given.

What is claimed is:

1. A system for determining optimal stimulation parameters for treating intractable epilepsy, the system adapted to:
impart deep brain stimulation to a patient's brain;
record EEG responses to the stimulation;
enable adjustment of one or more stimulation parameters to achieve an optimum EEG desynchronization state; and
select stimulation parameters that resulted in the optimum EEG desynchronization.

2. The system according to claim 1, wherein the system is further configured to induce an anti kindling effect.

3. The system according to claim 1, wherein the deep brain stimulation is anterior thalamic nucleus deep brain stimulation.

4. The system according to claim 1, wherein the deep brain stimulation is a centromedian thalamic nucleus deep brain stimulation.

5. The system according to claim 3, wherein the system is configured to impart the anterior thalamic nucleus deep brain stimulation involving Papez circuit.

6. The system according to claim 4, wherein the system is configured to impart the centromedian thalamic nucleus deep brain stimulation involving reticular activating system.

7. A method for determining optimal stimulation parameters for inducing EEG desynchronization for treating intractable epilepsy, the method comprising:
imparting deep brain stimulation to a patient's brain;
recording EEG responses to the stimulation;
enabling adjustment of one or more stimulation parameters to achieve an optimum EEG desynchronization state; and
selecting stimulation parameters that resulted in the optimum EEG desynchronization.

8. The method according to claim 7, the method further comprising inducing an anti kindling effect.

9. The method according to claim 7, wherein the method further comprises, imparting an anterior thalamic nucleus deep brain stimulation involving Papez circuit.

10. The method according to claim 7, wherein the method further comprises, imparting a centromedian thalamic nucleus deep brain stimulation involving reticular activating system.

11. A pulse width for inducing electroencephalography desynchronization. The pulse width in the range of 110 to 130 microseconds.

12. The pulse width according to claim 11, wherein the pulse width is in accordance with Weiss equation (pulse width×1/voltage).
